(19) United States
(12) Patent Application Publication
(10) Pub. No.: US 2012/0046631 A1
(43) Pub. Date: Feb. 23, 2012

(54) Convenience cleaning products pouch. Pouch for cleaning and disposable diaper industry

(76) Inventors: Joshua Pardue B., Los Angeles, CA (US); Branden Kakugawa, Laupahoehoe, HI (US)

(21) Appl. No.: 12/806,832

(22) Filed: Aug. 23, 2010

(51) Int. Cl. A61F 13/84; A61C 19/02

(52) U.S. Cl. 604/385.06; 206/369

(57) ABSTRACT

This Invention Marries Proximity And Convenience With The Placement Of A Hermetically Sealed Pouch Containing The Cleaning Products Of Baby Wipes And Disposable Trash Bag By Directly Attaching This Pouch To The Disposable Diaper.

### BACKGROUND OF INVENTION

One Day On Or About Nov. 21, 2007 I (Joshua Pardue) And Branden Kakugawa Were Thinking Of Ways Of How To Make Money Honestly. Joshua Told Me He Likes To Invent Different Things, So I Grabbed A Newspaper And We Started Looking At Different Products And How We Can Make Them Better Or More Convenient. When We Came Across An Advertisement Article About Diapers And Baby Wipes We Thought How Inconvenient It Was To Always Have To Buy Them Separate And Not Together. That's When We Came Up With An Idea To Put The Baby Wipes Some How Into The Diaper. And Then We Thought How Inconvenient It Was To Carry Around A Dirty Diaper With No Rubbish Can Around When You Needed It. Thus We Came Up With The Idea Of Adding A Small Disposal Trash Bag With The Baby Wipes, And Then We Came Up With The Idea To Create A Pouch To Hold These Two Products Together, So That You Have All The Convenience In One Little Diaper. Thus Helping The Consumer Not Having To Go Digging Through A Diaper Bag For The Baby Wipes And Not Ever Having To Worry Of Disposing Of It, Making It Very Easy And Convenient To Anyone Who Chooses To Use This Product.

### BRIEF SUMMARY OF INVENTION

The Utility Of This Invention Is Birthed Out Of An Inconvenience Created As A By-Product Of Another Convenience Industry; That Being The Disposable Diaper Industry. What To Do With The Mess? These Questions Are Address Generally With Baby Wipes (Moist Toilets) And Waste Management. But This Invention Addressed The Gap Of Inconvenience Between The Mess And The General Cleaning Remedies. (1) By Brining The Appropriate Cleaning (Wipes) And Disposable (Trash Bags) Products Directly Into The Hands Of The Consumer At The Moment Of Need! (2) By Placing These Supplies In A Hermetically Sealed Pouch Attached Directly To The Disposable Diaper. (Proximity)

### BRIEF DESCRIPTION OF SEVERAL VIEWS OF DRAWING

FIG. 1.

Shows The Pouch. The Key Elements Particular To The Claims Is That The Pouch Is Self-Contained, Separate From The Diaper, Hermetically Sealed To Retain The Moisture Of The Wipe Products And To Prevent Contamination Of The Enclosed Products. The Partition Are Not Absolute But Used To Depict Two Products. Size Is Not Absolute, Leaving Room For Various Manufacturing Considerations In Folding, Packing, And End Product Design.

FIG. 2.

Shows That The Pouch Has Easy Access To The Products Sealed Therein. The Type Of Seal And Unsealing Are Open To The Many Variables Of Manufacturing, Product Design, And Safety Consideration.

FIG. 3.

Depicts A Side View Of The Diaper Showing The Pouch As A Separate Product, Location Is Not Absolute In That Safety, Size, End Product, And Manufacturing Concerns Could Determine The Best Feature For The Claim Of Convenience.

FIG. 4.

Depicts The Diaper Open Inside Up, Again Showing The Pouch As A Separate Product. Location, Size, And Access Are Not Absolute Remaining Final Consideration Of Manufacturing.

FIG. 5(A).

Depicts The Transparent Front View Revealing The Pouch In Its Suggested Position, Between The Outer Shell And Inner Wadding. Also Locating The Pouch Seal Near The Waistband. This Description Is Not A Factual Absolute Or Manufacturing, But Is Intended To Display The Absolute Claim Of Easy Access, Proximity, And Convenience.

FIG. 5(B).

A Non-Transparent Front View.

FIG. 5(C).

A Transparent Inner View Depicting The General Suggested Location And The Pouch Separation From The Diaper Product.

FIG. 5(D).

The Same View As FIG. 3.

FIG. 6.

Depicts The Telescoping View Of The Multiple Layers Of The Diaper Including The Pouch.

FIG. 6(A). The Inner Moisture Barrier.

FIG. 6(B). Same As FIG. 6(A). Side View.

FIG. 6(C). The Wadding/Absorbant Material.

FIG. 6(D). Same As FIG. 6(C). Side View.

FIG. 6(E). The Outer Plastic Lining.

FIG. 6(F). The Same As FIG. 6(E). Side View.

FIG. 6(G). The Pouch.

FIG. 6(H). Same As FIG. 6(G). Side View.

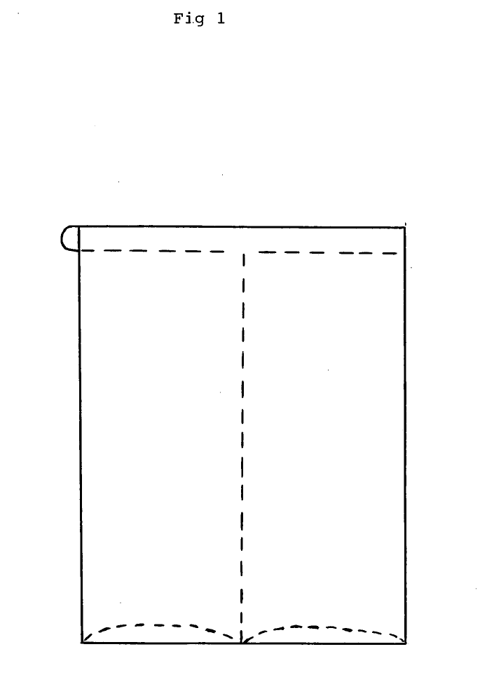

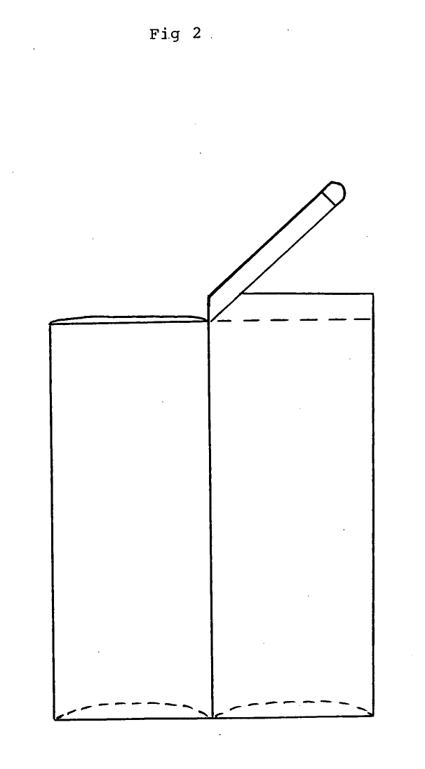

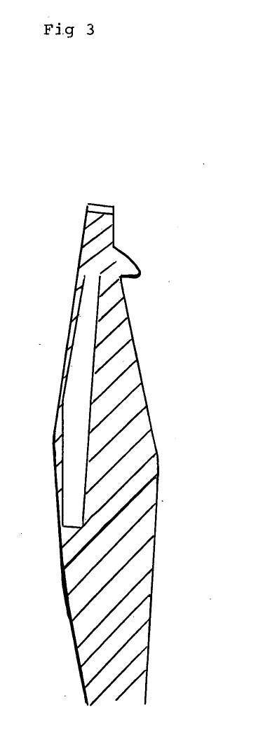

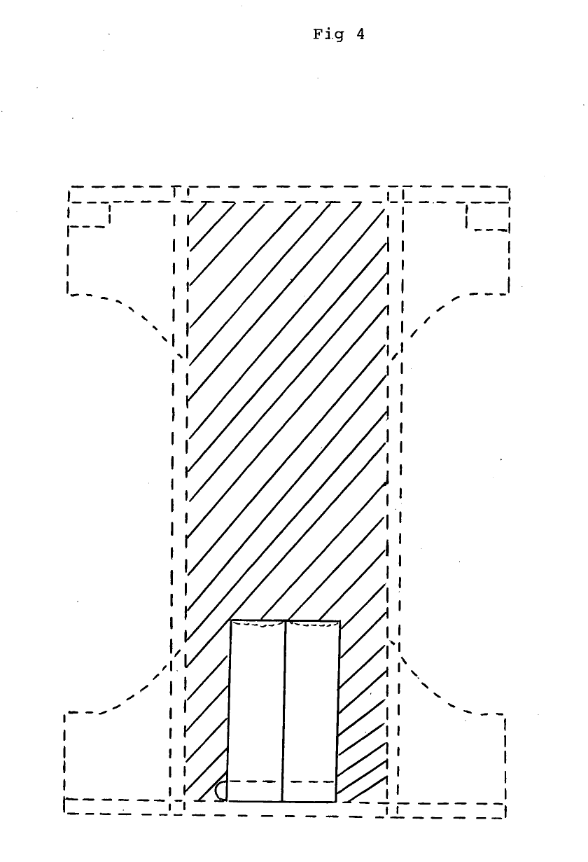

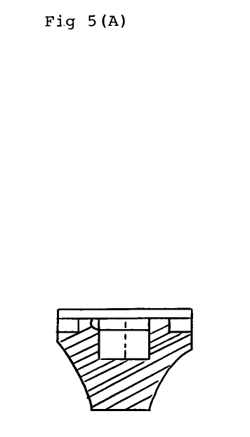

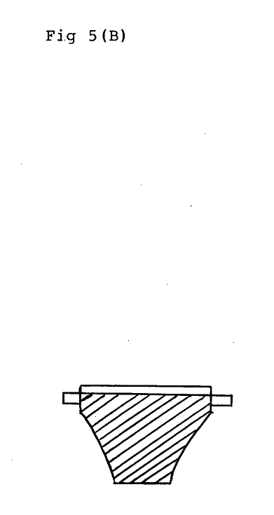

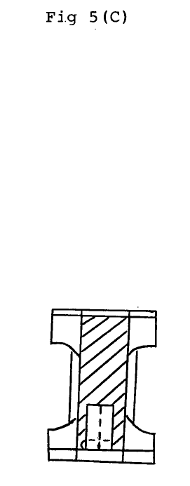

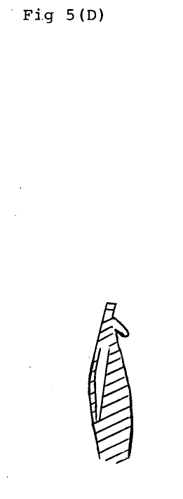

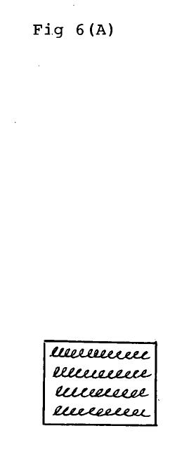

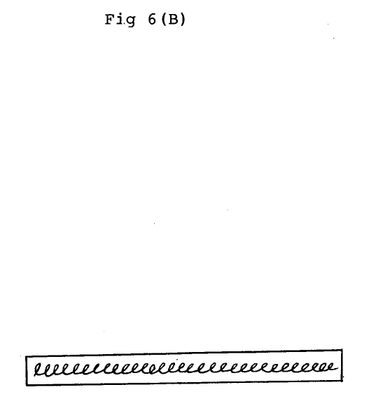

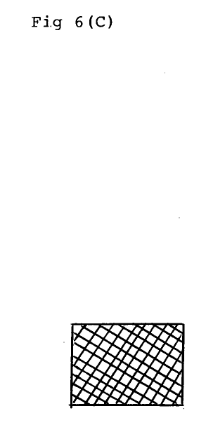

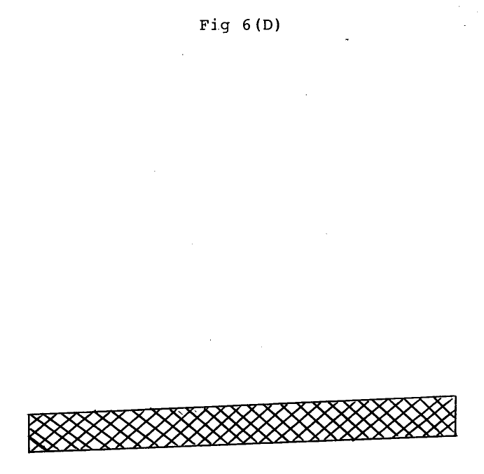

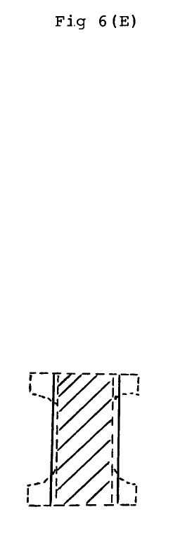

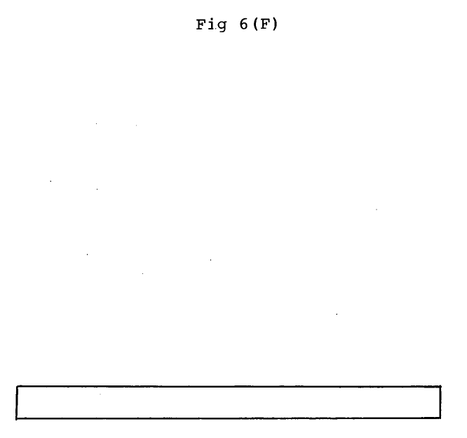

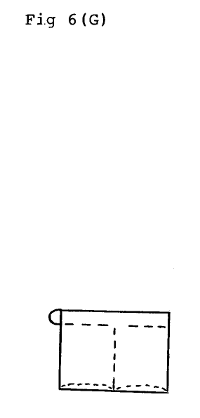

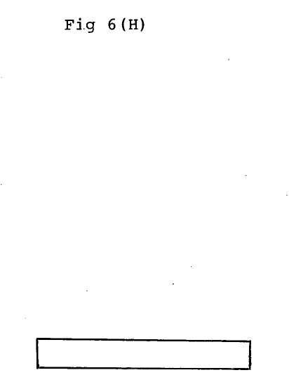

These Figures Again Are Only To Clarify The Pouch And Make No Representation As To The Actual Diaper Product Or The Absolute Of Manufacturing. The Claims Remain The Same.

### DETAILED DESCRIPTION OF THE INVENTION

The Convenience Products Pouch Is An Invention Of Convenience That Improves The Traditional Disposable Diaper By Adding Cleaning Products In Direct Proximity To The Need To Clean And Dispose Of The Waste. It Does This By Adding A Pouch Containing Moist Toilets (Known As Baby Wipes), And A Disposable Trash Bag Directly To The Disposable Diaper. These Products Are Contained In An Hermetically Sealed Pouch Built Into The Diaper Construction. The Location, Size, And Seal Cannot be Addressed By This Invention Because Manufacturing And Safety Concerns As Well As Folding Techniques For The Wipes And Trash Bag Could All Vary These Factors. The Claims Of This Invention That Are Not Variables Is The Cleaning Products Placed In A Pouch In Direct Proximity To The Need By Having It Attached To The Consumer When And Where Needed. The Pouch Is To Function As A Self-Contained Remedy To The Consumers' Concerns And Needs When Faced With The Aftermath Of A Soiled Diaper.

1. This Product Makes The Claim Of Proximity, Convenience, And Pouch.
The Pouch Is Added To A Disposable Diaper Containing Clean-Up Products To Address The Soiled Diaper. These Contained Products Are Baby Wipes And A Disposable Plastic Trash Bag.
The Pouch Is Hermetically Sealed And Is Separate And Distinct From The Diaper Itself Though Attached.
Proximity Is A Claim Of This Product In That It Is Attached Directly To The Diaper And Easily Accessible To The Consumer While Dealing With The Clean-Up.
Convenience Is The Functional Utility Of This Product By Addressing A Market Need With Its Pouch And Proximity.
Size, Location, Any Parts Of The Description Or Depiction That Address Those Issues Were Made For The Soiled Purpose Of Clarification Of The Claim Of This Invention.
